(19) United States
(12) Patent Application Publication
(10) Pub. No.: US 2024/0410034 A1
(43) Pub. Date: Dec. 12, 2024

(54) CYCLIC AMINE MONOAMIDES FOR EXTRACTING URANIUM(VI) AND PLUTONIUM(IV) AND FOR SEPARATING THEM WITHOUT REDUCING PLUTONIUM(IV)

(71) Applicants: COMMISSARIAT À L'ÉNERGIE ATOMIQUE ET AUX ÉNERGIES ALTERNATIVES, Paris (FR); ORANO RECYCLAGE, CHATILLON (FR); ELECTRICITE DE FRANCE, PARIS (FR)

(72) Inventors: Cécile MARIE, SAINT-DIDIER (FR); Audrey BEILLARD, AVIGNON (FR); Valérie ALOIN, LAUDUN (FR); Christian SOREL, VILLENEUVE-les-AVIGNON (FR)

(21) Appl. No.: 18/702,559

(22) PCT Filed: Oct. 14, 2022
(86) PCT No.: PCT/FR2022/051946

§ 371 Date: Apr. 18, 2024

(30) Foreign Application Priority Data
Oct. 21, 2021 (FR) FR2111198

(51) Int. Cl. C22B 60/02; C07D 211/16; C22B 3/36; C22B 7/00; C22B 60/04; G21C 19/38; G21F 9/12

(52) U.S. Cl. CPC C22B 60/026; C07D 211/16; C22B 3/362; C22B 7/007; C22B 60/0239; C22B 60/04; G21C 19/38; G21F 9/125

(57) ABSTRACT

Uses of cyclic amine monoamides for extracting uranium(VI) and/or plutonium(IV) from an acidic aqueous solution, as well as for totally or partially separating uranium(VI) from plutonium(IV) from an acidic aqueous solution. A method for treating an aqueous solution resulting from the dissolution of spent nuclear fuel in nitric acid to extract, separate and decontaminate uranium(VI) and plutonium(IV) in a single cycle and without resorting to any operation of reducing plutonium(IV), and wherein a cyclic amine monoamide or a mixture of cyclic amine monoamides is used as extractant. The cyclic amine monoamides have formula (I):

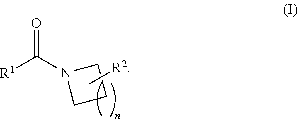

### TECHNICAL FIELD

The invention is directed to the field of spent nuclear fuel treatment.

More particularly, it is directed to the use of cyclic amine monoamides to extract uranium(VI) and/or plutonium(IV) from an acidic aqueous solution and, in particular, from an aqueous solution resulting from the dissolution of a spent nuclear fuel in nitric acid.

It is also directed to the use of these cyclic amine monoamides to separate, totally or partially, uranium(VI) from plutonium(IV) from an acidic aqueous solution and, in particular, from an aqueous solution resulting from the dissolution of a spent nuclear fuel in nitric acid.

It is also directed to a method for treating an aqueous solution resulting from the dissolution of a spent nuclearfuel in nitric acid, which makes it possible to extract, separate and decontaminate uranium(VI) and plutonium(IV) present in this solution in a single cycle and without resorting to any operation of reducing plutonium(IV), and wherein one of these cyclic amine monoamides or a mixture thereof is used as an extractant.

It is further directed to cyclic amine monoamides likely to be used in the aforementioned uses and method.

The invention finds application in the treatment of uranium-based (especially uranium oxides-based, known as UOX fuels), or uranium and plutonium-based (especially mixed uranium and plutonium oxides-based, known as MOX fuels) spent nuclear fuels.

### State of Prior Art

The PUREX method, which is used in all the spent nuclear fuel treatment plants in the world (La Hague in France, Rokkasho in Japan, Sellafield in the United Kingdom, etc.), uses tri-n-butyl phosphate (or TBP) as an extractant to recover uranium(VI) and plutonium(IV) by liquid-liquid extraction from aqueous solutions resulting from the dissolution of these fuels in nitric acid.

In this method, TBP is used as a 30% (v/v) solution in an aliphatic diluent of the hydrogenated tetrapropylene (or TPH) type or the like. This organic solution is commonly referred to as a “solvent” in the field considered.

Uranium and plutonium are recovered by the PUREX method in several cycles:
a first cycle of purifying uranium(VI) and plutonium(IV) (called “1CUPu”), which aims at decontaminating these elements with respect to minor actinides (neptunium, americium and curium) as well as with respect to fission and activation products, with partitioning uranium(VI) and plutonium(IV) into two aqueous flows from this first cycle onwards;
a second cycle of purifying uranium(VI) (called “2CU”), which aims at enhancing decontamination of this element to achieve the specifications defined by ASTM standards for uranium as a finished product; and
a second cycle and, in the case of some plants, a third cycle of purifying plutonium(IV) (called “2CPu” and “3CPu” respectively), which aim at enhancing decontamination of this element to achieve the specifications defined by the ASTM standards for plutonium as a finished product, and to concentrate it before being converted into oxide PuO₂.

The performance of the PUREX method is satisfactory, and feedback from the plants that have implemented it since it started up has been positive.

However, the use of TBP has limitations that are opposite to the possibility of achieving, with this extractant, the objectives of simplicity, compactness and improved safety set for future spent nuclear fuel treatment plants.

The main limitation is that the partitioning of uranium and plutonium into two aqueous flows requires the plutonium(IV) to be reduced to plutonium(III) because, with TBP, the separation factor between uranium(VI) and plutonium(IV) is insufficient, whatever the acidity of the aqueous solution used for partitioning. As a result, the use of TBP requires the use of reducing and anti-nitrous agents in large amounts, which generate unstable and reactive species by degradation and are therefore restrictive in terms of safety.

Some work has therefore been carried out to provide extractants that can quantitatively co-extract uranium and plutonium from an aqueous solution resulting from the dissolution of a spent nuclear fuel in nitric acid, and then ensure a total or partial separation of these two elements without having to reduce plutonium(IV) to plutonium(III).

Thus there has been suggested the use of:
symmetrical N,N-dialkylamides, such as N,N-di(2-ethylhexyl)-3,3-dimethylbutanamide (or DEHDMBA), in International PCT application WO 2017/017207, hereinafter reference [1],
dissymmetric N,N-dialkylamides, such as:
   N-methyl-N-octyl-2-ethylhexanamide (or MOEHA) or N-decyl-N-methyl-2-ethylhexanamide (or MDEHA), in International PCT application WO 2017/017193, hereinafter reference [2],
   N-methyl-N-nonyl-2-methylhexanamide (or MNMHA) or N-methyl-N-hexyl-2-propylheptanamide (or MHPHepA) in International application PCT WO 2018/138441, hereinafter reference [3], and
tetraalkylated carbamides, such as N,N,N′,N′-tetra-n-butylurea (or TBU), or trialkylated carbamides, such as N,N,N′-tri-n-octylurea (or TrOU) in International application PCT WO 2019/002788, hereinafter reference [4].

All these extractants and, in particular, the dissymmetric N,N-dialkylamides of references [2] and [3], were found to exhibit very interesting performance.

Furthermore, within the scope of a study published in 1960 in J. Phys. Chem, 64, 12, 1863-1866, hereinafter reference [5], and aimed at assessing the ability of numerous N,N-disubstituted amides to extract uranium(VI) and plutonium(IV) from highly acidic aqueous phases of nitric acid (3 mol/L and 6 mol/L), T. H. Siddall studied that of two cyclic amine monoamides, namely N-hexanoylpiperidine and N-2-ethylhexanoylpiperidine, at a concentration of 0.5 mol/L in toluene.

Also reported by H. Jing-Tian et al. (Journal of Radioanalytical and Nuclear Chemistry 1999, 241, 215-217, hereinafter reference [6]) are data on the extraction of uranium(VI) by a cyclic amine monoamide, herein N-octanoylpyrrolidine, at a concentration of 0.3 mol/L in toluene.

Reference [5] shows that, although quite similar in structure, the two piperidine ring monoamides give contradictory extraction results under the same operating conditions, regardless of whether at an acidity of 3 mol/L or 6 mol/L nitric acid, since at these two acidities, N-hexanoylpiperidine extracts plutonium(IV) better than uranium(VI), whereas N-2-ethylhexanoylpiperidine extracts uranium(VI) better than plutonium(IV).

Reference [6] states that when used at a concentration of 0.3 mol/L in toluene, N-octanoylpyrrolidine very weakly extracts uranium(VI) from a 3 mol/L aqueous solution of nitric acid with a distribution coefficient, Dᵤ, of less than 1 at 25° C.

Furthermore, in view of the above, data reported in references [5] and [6] do not particularly encourage the use of cyclic amine monoamides for quantitatively co-extracting uranium(VI) and plutonium(IV) from a highly acidic aqueous solution of nitric acid, they do not in any way suggest the possibility of using this type of compound to separate uranium(VI) from plutonium(IV), once these have been co-extracted from such a solution, without resorting to a reduction of plutonium(IV) to plutonium(III).

Within the scope of their work, the inventors have noticed that monoamides with an azetidine, pyrrolidine or piperidine ring substituted with one or more alkyl or alkoxy groups have extracting properties such that:
in the presence of a highly acidic aqueous phase of the type found in aqueous solutions resulting from the dissolution of spent nuclear fuels in nitric acid, they lead to distribution coefficients for uranium(VI) and plutonium(IV) which are suitable for allowing a quantitative co-extraction of these two elements from this aqueous phase, and
in the presence of a moderately acidic aqueous phase, they lead to U(VI)/Pu(IV) separation factors suitable for separating uranium(VI) from plutonium(IV) without it being necessary to reduce plutonium(IV) to plutonium(III), this separation being total or partial according to what is desired.

The present invention is based on these experimental findings.

### DISCLOSURE OF THE INVENTION

First, one object of the invention is therefore the use of a monoamide or a mixture of monoamides of formula (I) hereinafter:

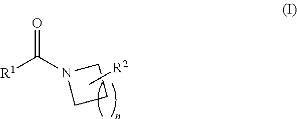

wherein:
n=1, 2 or 3;
R¹ is a straight or branched chain alkyl group comprising from 4 to 12 carbon atoms; and
R² is a straight or branched chain alkyl or alkoxy group, the chain of which is optionally interrupted one or more times by an oxygen atom and the total number of carbon and, if applicable, oxygen atoms of which is between 3 and 10;
   for extracting uranium(VI) and/or plutonium(IV) from an acidic aqueous solution with a pH of less than 0.

In the foregoing and what follows, the phrases “from . . . to . . . ”, “ranging from . . . to . . . ” and “between . . . and . . . ” are equivalent and mean that the limits are included.

Furthermore:
by “straight or branched chain alkyl group comprising from 4 to 12 carbon atoms”, it is meant any alkyl group whose chain may or may not comprise one or more identical or different branches, and whose number of carbon atoms is equal to 4, 5, 6, 7, 8, 9, 10, 11 or 12; whereas
by “straight or branched chain alkyl or alkoxy group, the chain of which is optionally interrupted one or more times by an oxygen atom and the total number of carbon and, if applicable, oxygen atoms of which is between 3 and 10”, it is meant any alkyl or —O— alkyl group, the chain of which may or may not comprise one or more identical or different branches, and one or more insertions of an oxygen atom between two carbon atoms, and has a total number of carbon and oxygen atoms (if one or more oxygen atoms are present) equal to 3, 4, 5, 6, 7, 8, 9 or 10.

In accordance with the invention, uranium(VI) and/or plutonium(IV) are preferably extracted from the acidic aqueous solution by liquid-liquid extraction, i.e. by bringing this aqueous solution into contact with an organic solution comprising the monoamide or the mixture of monoamides in an organic diluent, and then separating the aqueous and organic solutions from each other.

In this case, the organic solution comprises, preferably, from 1 mol/L to 2 mol/L and, even more preferably, from 1 mol/L to 1.3 mol/L of the monoamide or of the mixture of monoamides, preference being given to a concentration of 1.2 mol/L.

The acidic aqueous solution is preferably an aqueous solution resulting from the dissolution of a spent nuclearfuel in nitric acid, i.e. an aqueous solution typically comprising from 3 mol/L to 6 mol/L of nitric acid.

In the foregoing and what follows, by “organic diluent”, it is meant any apolar hydrocarbon or mixture of apolar, aliphatic and/or aromatic hydrocarbons, the use of which has been provided for dissolving lipophilic extractants. By way of example of such a diluent, mention may especially be made of n-dodecane, hydrogenated tetrapropylene (TPH), kerosene and isoparaffinic diluents such as those marketed by TotalEnergies under the references Isane™ IP-185 and Isane™ IP-175.

In addition to be capable of quantitatively extracting uranium(VI) and plutonium(IV) from an acidic aqueous solution with a pH of less than 0, the monoamides of formula (I) hereinabove have been shown subsequently to make it possible to separate uranium and plutonium thus co-extracted from each other, without having to reduce the plutonium(IV) to plutonium(III), wherein this separation can be:
either a total separation of uranium(VI) and plutonium(IV), that is wherein two aqueous solutions are obtained, one comprising plutonium(IV) without uranium(VI) and the other comprising uranium(VI) without plutonium (IV);
or a partial separation of uranium(VI) and plutonium(IV), that is wherein two aqueous solutions are obtained, one comprising a mixture of plutonium(IV) and uranium(VI) and the other comprising uranium(VI) without plutonium(IV).

Thus, another object of the invention is the use of a monoamide or a mixture of monoamides of formula (I) hereinabove, to totally or partially separate uranium(VI) from plutonium(IV) from an acidic aqueous solution with a pH of less than 0, which use comprises:
a) a co-extraction of uranium(VI) and plutonium(IV) from the aqueous solution, this co-extraction comprising at least one contacting of the aqueous solution with an organic solution S1 comprising a monoamide or a mixture of monoamides of formula (I) in an organic diluent, followed by a separation of the aqueous and organic solutions from each other;
b) a stripping of plutonium, in the +IV oxidation state, and of a fraction of the uranium(VI) from the organic solution resulting from step a), this stripping comprising at least one contacting of the organic solution with an acidic aqueous solution with a pH greater than 0 and, better still, greater than 0.3, followed by a separation of the organic and aqueous solutions from each other; and
c) a re-extraction of all or part of the uranium(VI) fraction present in the aqueous solution resulting from step b), this re-extraction comprising at least one contacting of the aqueous solution with an organic solution S2 comprising the monoamide or the mixture of monoamides in the organic diluent, followed by a separation of the aqueous and organic solutions from each other;
   whereby an aqueous solution comprising either plutonium(IV) without uranium(VI), or a mixture of plutonium(IV) and uranium(VI), and an organic solution comprising uranium(VI) without plutonium(IV) are obtained.

Organic solutions S1 and S2 comprise, preferably, from 1 mol/L to 2 mol/L and, better still, from 1 mol/L to 1.3 mol/L of the monoamide or of the mixture of monoamides, most preferably a concentration of 1.2 mol/L.

The aqueous solution with a pH of less than 0 is preferably an aqueous solution resulting from the dissolution of a spent nuclear fuel in nitric acid, i.e. an aqueous solution typically comprising from 3 mol/L to 6 mol/L of nitric acid, in which case the acidic aqueous solution with a pH above 0 is preferably a solution comprising from 0.1 mol/L to 0.5 mol/L of nitric acid.

Uranium present in the organic solution resulting from step c) can then be extracted from this phase by contacting the organic solution with an acidic aqueous solution with a pH of at least 1.3, such as a solution comprising at most 0.05 mol/L of nitric acid, and then separating the organic and aqueous solutions from each other.

In addition to have the aforementioned properties, the monoamides of formula (I) hereinabove have been shown to lead to organic phases whose dynamic viscosity and uranium(VI) loading capacity are entirely compatible with their use in a method for treating spent nuclear fuels.

Given this combination of properties, these monoamides have made it possible to develop a method for treating an aqueous nitric solution resulting from the dissolution of a spent nuclear fuel which, while being as effective as the PUREX method in terms of recovering and decontaminating uranium and plutonium present in such a solution, is free of any plutonium-reducing stripping operation and includes only a single treatment cycle.

Accordingly, another object of the invention is a method for treating in one cycle an aqueous solution resulting from the dissolution of a spent nuclear fuel in nitric acid, the aqueous solution comprising uranium(VI), plutonium(IV), americium(III), curium(III) and fission and activation products including ruthenium and technetium, the cycle comprising:
a) a co-extraction of uranium(VI) and plutonium(IV) from the aqueous solution, this co-extraction comprising at least one contacting, in an extractor, of the aqueous solution with an organic solution S1 comprising a monoamide or a mixture of monoamides of formula (I) in an organic diluent, followed by a separation of the aqueous and organic solutions from each other;
b) a decontamination of the organic solution issued from step a) with respect to americium(III), curium(III) and fission and activation products, this decontamination comprising at least one contacting, in an extractor, of the organic solution with an aqueous solution comprising from 0.5 mol/L to 6 mol/L of nitric acid, followed by a separation of the organic and aqueous solutions from each other;
c) a partitioning of uranium(VI) and plutonium(IV) present in the organic solution issued from step b) into an aqueous solution comprising either plutonium(IV) without uranium(VI), or a mixture of plutonium(IV) and uranium(VI), and an organic solution comprising uranium(VI) without plutonium(IV), the partitioning comprising:
   c₁) a stripping of plutonium, in the +IV oxidation state, and of a uranium(VI) fraction from the organic solution issued from step b), this stripping comprising at least one contacting, in an extractor, of the organic solution with an aqueous solution comprising from 0.1 mol/L to 0.5 mol/L of nitric acid, followed by a separation of the organic and aqueous solutions from each other;
   c₂) a re-extraction of all or part of the uranium(VI) fraction present in the aqueous solution issued from step c₁), this re-extraction comprising at least one contacting, in an extractor, of the aqueous solution with an organic solution S2 comprising the monoamide or the mixture of monoamides in the organic diluent, followed by a separation of the aqueous and organic solutions from each other;
d) a decontamination of the organic solution issued from step c₁) with respect to technetium, this decontamination comprising:
   d₁) a stripping of technetium, in the +IV oxidation state, from the organic solution issued from step c₁), this stripping comprising at least one contacting, in an extractor, of the organic solution with an aqueous solution comprising from 0.1 mol/L to 3 mol/L of nitric acid and at least one reducing agent capable of reducing technetium from the +VII oxidation state to the +IV oxidation state, followed by a separation of the organic and aqueous solutions from each other;
   d₂) a re-extraction of the uranium(VI) fraction present in the aqueous solution issued from step d₁), this re-extraction comprising at least one contacting, in an extractor, of the aqueous solution with an organic solution S3 comprising the monoamide or the mixture of monoamides in the organic diluent, followed by a separation of the aqueous and organic solutions from each other;
e) a stripping of uranium(VI) from the organic solution issued from step d₁), this stripping comprising at least one contacting, in an extractor, of the organic solution with an aqueous solution comprising at most 0.05 mol/L of nitric acid, followed by a separation of the organic and aqueous solutions from each other; and
f) optionally, a regeneration of the organic solution from step e);
   whereby a first and a second aqueous solution decontaminated with respect to americium(III), curium(III) and fission and activation products are obtained, the first aqueous solution comprising either plutonium(IV) without uranium(VI), or a mixture of plutonium(IV) and uranium(VI), and the second aqueous solution comprising uranium(VI) without plutonium(IV).

In accordance with the invention, the organic solutions S1, S2 and S3 comprise, preferably, from 1 mol/L to 2 mol/L and, better still, from 1 mol/L to 1.3 mol/L of the monoamide or of the mixture of monoamides, most preferably a concentration of 1.2 mol/L.

As previously indicated, the aqueous solution used in step b) may comprise from 0.5 mol/L to 6 mol/L of nitric acid. However, it is preferred that this aqueous solution comprise from 4 mol/L to 6 mol/L of nitric acid so as to facilitate the stripping of ruthenium and technetium from the organic solution issued from step a). In this case, step b) advantageously also comprises deacidifying the organic solution, this deacidifying comprising at least one contacting of the organic solution with an aqueous solution comprising from 0.1 mol/L to 1 mol/L and, even better, 0.5 mol/L of nitric acid, followed by a separation of the organic and aqueous solutions from each other.

In accordance with the invention, the contacting of the organic and aqueous solutions in the extractor in which step c₁) takes place comprises circulating these solutions in a flow rate ratio O/A which is advantageously greater than 1, preferably equal to or greater than 3 and, still more advantageously, equal to or greater than 5 so as to obtain a concentrating stripping of plutonium, i.e. a stripping of plutonium which leads to an aqueous solution in which the plutonium concentration is higher than the plutonium concentration in the organic solution from which it is stripped.

The reducing agent(s) present in the aqueous solution used in step d₁) is (are) preferably selected from uranous nitrate (also known as “U(IV)”), hydrazinium nitrate (also known as “hydrazine nitrate”), hydroxylammonium nitrate (also known as hydroxylamine nitrate), acetaldoxime, hydroxyiminoalkanoic acids (such as 6-hydroxyiminohexanoic acid) and mixtures thereof, such as a mixture of uranous nitrate and hydrazinium nitrate, a mixture of uranous nitrate and hydroxylammonium nitrate or a mixture of uranous nitrate and acetaldoxime, preference being given to a mixture of uranous nitrate and hydrazinium nitrate or a mixture of uranous nitrate and hydroxylammonium nitrate, which are preferably used at a concentration ranging from 0.1 mol/L to 0.3 mol/L and, typically, 0.2 mol/L.

Furthermore, step d₁), which can be carried out at room temperature, is, however, preferably carried out at a temperature ranging from 30° C. to 40° C. and, better still, 32° C. so as to promote the stripping kinetics of technetium while limiting as far as possible the phenomena of reoxidation of this element in the aqueous phase. The extractor in which step d₁) takes place is therefore preferably heated to a temperature of between 30° C. and 40° C.

In accordance with the invention, step d₂) preferably additionally comprises acidifying the aqueous solution issued from step d₁), this acidifying comprising adding nitric acid to the extractor in which step d₂) takes place in order to bring the concentration of nitric acid in the aqueous solution to a value at least equal to 2.5 mol/L.

Step e) may be carried out at room temperature. However, it is preferably carried out at a temperature of between 40° C. and 50° C., again to promote stripping of the uranium. The extractor in which step e) takes place is therefore preferably heated to a temperature of between 40° C. and 50° C.

Whatever the temperature at which step e) is carried out, the contacting of the organic and aqueous solutions in the extractor in which this step takes place comprises circulating these solutions in an O/A flow rate ratio greater than 1 so as to obtain a concentrating stripping of uranium, i.e. a stripping of uranium which leads to an aqueous solution in which the concentration of uranium is higher than the uranium concentration in the organic solution from which it is stripped.

Preferably, the method comprises step f) of regenerating the organic solution from step e), this regenerating preferably comprising at least washing the organic solution with a basic aqueous solution, followed by at least washing the organic solution with an aqueous nitric acid solution.

In which case, the organic solution thus regenerated is then advantageously divided into a first and a second fraction, the first fraction forming the organic solution S1 used in step a) and the second fraction forming the organic solution S2 used in step c₂).

The method described in this invention has a number of advantages. Indeed:
uranium stripping is easier to implement than in the PUREX method, since it can be carried out both at room temperature and while hot, and using an O/A flow rate ratio greater than 1, which makes it possible to strip uranium in a concentrating manner, which is not possible in the PUREX method;
since it does not involve any plutonium reduction reaction and therefore eliminates any risk of plutonium reoxidation, plutonium stripping is also easier to implement than the PUREX method and can be carried out in a more concentrating manner than the latter; these advantages are all the more important as future spent nuclear fuel treatment plants will have to process fuels that are richer in plutonium (such as MOX fuels from light water or fast neutron reactors) than the fuels currently treated;
the degradation products (by hydrolysis and radiolysis) of monoamides are less troublesome than those of TBP because they are soluble in water and do not form complexes likely to retain plutonium;
the solubility of monoamides in the aqueous phase is typically 100 to 200 times lower than that of TBP, which makes it possible to contemplate the elimination or, at the very least, a reduction in the number of washes with organic diluent of the aqueous solutions resulting from the method of the invention with respect to those provided for in the PUREX method;
since monoamides and their degradation products comprise only carbon, hydrogen, oxygen and nitrogen atoms, they can be completely incinerated and therefore do not produce any harmful secondary waste, unlike TBP and its degradation products.

As previously indicated, in formula (I) above, n can be equal to 1, 2 or 3.

In which case, the monoamide of formula (I) can have one of the particular formulae (Ia), (Ib) or (Ic) below:

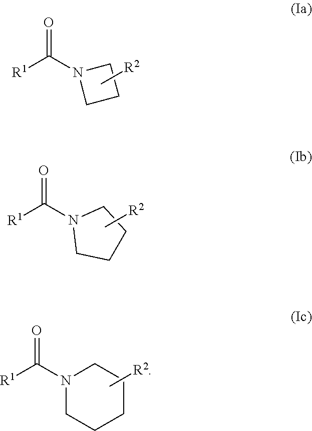

In accordance with the invention, n is preferably equal to 2 or 3 so that the monoamide of formula (I) preferably has one of the particular formulae (Ib) and (Ic).

Furthermore, R¹ is advantageously a straight or branched chain alkyl group comprising from 6 to 10 carbon atoms and, still better, a branched chain alkyl group such as a 1-methylpentyl, 1-methylhexyl, 1-methylheptyl, 1-methyloctyl, 1-methylnonyl, 1-ethylpentyl, 1-ethylhexyl, 1-ethylheptyl, 1-ethyloctyl, 1-propylbutyl, 1-propylpentyl, 1-propylhexyl, 1-propylheptyl, 2-ethylhexyl, 2-ethylheptyl, 2-ethyloctyl, etc., group. Among these, most preferably it is a 1-ethylpentyl or 1-propylbutyl group.

As for R², it is advantageously a straight or branched chain alkyl group comprising from 4 to 10 carbon atoms and, even better, a straight chain alkyl group, i.e. an n-butyl, n-pentyl, n-hexyl, n-heptyl, n-octyl, n-nonyl or n-decyl group. Of these, most preferably, it is an n-hexyl, n-heptyl or n-octyl group.

Monoamides which meet these preferences are especially:
N-(2-heptylpyrrolidinyl)-(2-ethyl)hexanamide, hereinafter referred to as EHPyr2, which has the particular formula (Ib) wherein R¹ represents a 1-ethylpentyl group while R² represents an n-heptyl group which is carried by the carbon atom located in position 2 of the pyrrolidine ring;
N-(2-hexylpiperidinyl)-(2-ethyl)hexanamide, hereinafter referred to as EHPip2, which has the particular formula (Ic) wherein R¹ represents a 1-ethylpentyl group while R² represents an n-hexyl group which is carried by the carbon atom located in position 2 of the piperidine ring;
N-(3-hexylpiperidinyl)-(2-ethyl)hexanamide, hereinafter referred to as EHPip3, which has the particular formula (Ic) wherein R¹ represents a 1-ethylpentyl group while R² represents an n-hexyl group which is carried by the carbon atom located in position 3 of the piperidine ring;
N-(4-hexylpiperidinyl)-(2-ethyl)hexanamide, hereinafter referred to as EHPip4, which has the particular formula (Ic) wherein R¹ represents a 1-ethylpentyl group while R² represents an n-hexyl group which is carried by the carbon atom located in position 4 of the piperidine ring; and
N-(3-hexylpiperidinyl)-(2-propyl)pentanamide, hereinafter referred to as PPPip3, which has the particular formula (Ic) wherein R¹ represents a 1-propylbutyl group while R² represents an n-hexyl group which is carried by the carbon atom located in position 3 of the piperidine ring.

Among these, the monoamide EHPyr2 is most preferably used because, in addition to very effectively co-extracting uranium(VI) and plutonium(IV) from a highly acidic aqueous phase such as an aqueous phase comprising from 3 mol/L to 6 mol/L of nitric acid, it has a particularly high selectivity for uranium(VI) towards plutonium(IV) in the presence of a moderately acidic aqueous phase, such as an aqueous phase comprising from 0.1 mol/L to 0.5 mol/L of nitric acid.

Some of the monoamides of formula (I) are known from the state of the art. On the other hand, others have not, to the best of the Inventors' knowledge, been described in the literature.

The invention further relates to these new monoamides, which are:
N-(2-heptylpyrrolidinyl)-(2-ethyl)hexanamide,
N-(2-hexylpiperidinyl)-(2-ethyl)hexanamide,
N-(3-hexylpiperidinyl)-(2-ethyl)hexanamide,
N-(4-hexylpiperidinyl)-(2-ethyl)hexanamide, and
N-(3-hexylpiperidinyl)-(2-propyl)pentanamide.

Further characteristics and advantages of the invention will be apparent from the following description.

It goes without saying, however, that this further description is given solely by way of illustration of the object of the invention and should not in any way be interpreted as a limitation of this object.

### BRIEF DESCRIPTION OF THE FIGURE

FIG. 1 illustrates a schematic diagram of a method for treating an aqueous solution resulting from the dissolution of a spent nuclear fuel in nitric acid in accordance with the invention; in this FIGURE, rectangles 1 to 7 represent multi-step extractors such as those conventionally used in the treatment of spent nuclear fuel (mixer-settlers, pulsed columns or centrifugal extractors); the organic phases are symbolised by solid lines while the aqueous phases are symbolised by dotted lines.

### DETAILED DESCRIPTION OF PARTICULAR EMBODIMENTS

### I—Synthesis of Monoamides of Formula (I)

The monoamides of formula (I) can be synthesised by two different procedures, referred to below as A and B respectively.

### Procedure A:

This is based on the reaction of a carboxylic acid of the formula: R¹—COOH wherein R¹ has the same meaning as in formula (I) with a cyclic amine of the formula:

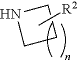

wherein n and R² have the same meaning as in formula (I).

This reaction can, for example, be conducted as follows: a solution comprising 1-hydroxybenzotriazole monohydrate (or HOBt—1.20 eq.) and N,N-dicyclohexylcarbodiimide (or DCC—1.20 eq.) in 2-methyltetrahydrofuran (0.2 mol/L) is stirred at room temperature for 10 min until completely dissolved. Carboxylic acid (1.00 eq.) is then added to the mixture. The resulting solution is stirred for 10 min and then a solution of the cyclic amine (1.00 eq.), optionally in salt form, in 2-methyltetrahydrofuran is added. The resulting suspension is stirred overnight at room temperature and then filtered on Celite™. The filtrate is washed twice with a saturated sodium bicarbonate solution, and then once with a saturated sodium chloride solution. The solution is finally dried with magnesium sulphate, filtered and then concentrated in vacuo.

### Procedure B:

This is based on the reaction of an acid halide of the formula: R¹—C(O)X wherein X represents a halogen atom, for example a chlorine atom, and R¹ has the same meaning as in formula (I) with a cyclic amine of the formula as represented hereinabove.

This reaction can be conducted as follows: triethylamine (1.50-4.00 eq.) is added to a solution of the cyclic amine (1.00 eq.), optionally in salt form, in dichloromethane (or DCM). The resulting solution is stirred at room temperature for 10 min then acid halide (1.50 eq.) is added. After stirring for two hours at room temperature, water is added to the reaction medium. The aqueous phase is then extracted 3 times with dichloromethane. The organic phases are combined and washed with a saturated sodium bicarbonate solution, and then with a saturated sodium chloride solution, dried with magnesium sulphate, filtered and concentrated in vacuo.

### Application of Procedures A and B to the Synthesis of Particular Monoamides of Formula (I):

The different monoamides of formula (I) whose synthesis is reported hereinafter may possess several stereogenic centres leading to the formation of diastereoisomers. These diastereoisomers are visible in ¹H or ¹³C NMR. In addition, each diastereomer may have rotamers, which are also visible in NMR. To aid understanding of their NMR analysis, the percentage proportion of each peak has been added below where possible.

### Synthesis of Monoamide EHPip2

Monoamide EHPip2, which has the particular formula hereinafter:

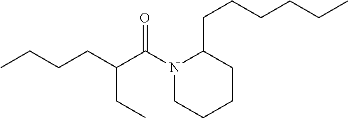 [SMILES: CCCCCCC1CCCCN1C(=O)C(CC)CCCC]

is synthesised using procedure B wherein 2-ethylhexanoyl chloride (3.00 mL, 17.4 mmol, 1.05 eq.) as the acid chloride, 2-hexylpiperidine hydrochloride (3.40 g, 16.5 mmol, 1.00 eq.) as the cyclic amine salt, triethylamine (5.6 mL, 41.3 mmol, 2.50 eq.) and DCM (165 mL) are used.

This yielded 4.42 g (15.7 mmol) of EHPip2 as a yellow oil. Yield: 95%.

¹H NMR (CDCl₃, 400 MHz): δ (ppm): 4.92-4.83 (m, 1H, 56%), 4.67-4.63 (m, 1H, 22%), 4.63-4.57 (m, 1H, 22%), 4.07-3.97 (m, 1H, 44%), 3.87-3.81 (m, 1H, 28%), 3.81-3.74 (m, 1H, 28%), 3.07 (t, J=13.7 Hz, 1H, 28%), 3.06 (t, J=13.8 Hz, 1H, 28%), 2.64-2.55 (m, 1H, 44%+1H, 100%), 1.76-1.54 (m, 8H), 1.51-1.36 (m, 4H), 1.34-1.12 (m, 12H), 0.95-0.82 (m, 100%)

¹³C NMR (CDCl₃, 101 MHz): δ (ppm): 174.92 (C═O, 22%), 174.87 (C═O, 22%), 174.6 (C═O, 56%), 53.0 (C═O, 56%), 52.9 (C═O, 44%), 48.1, 48.0, 42.8 (78%), 42.6 (22%), 40.99 (44%), 40.95 (56%), 36.68, 36.66, 33.1, 32.9, 32.6, 32.3, 32.02, 31.98, 31.93, 31.90, 30.63, 30.57, 30.5, 30.2, 30.0, 29.9, 29.6, 29.52, 29.48, 29.45, 29.4, 28.6, 28.5, 26.9, 26.83, 26.76, 26.45, 26.41, 26.39, 26.3, 26.2, 26.00, 25.95, 25.7, 23,2, 23.12, 23.07, 23.06, 22.74, 22.71, 19.35 (44%), 19.34 (56%), 14.23, 14.19, 12.7, 12.5, 12.20, 12.16

IR (cm⁻¹): 2955, 2924, 2872, 2855, 1636, 1437, 1250, 1217

HRMS (CI+): Theoretical exact mass for C₁₉H₃₈NO⁺ [M+H]⁺: 296.2953; experimental: 296.2963.

### Synthesis of Monoamide EHPip3

The monoamide EHPip3, which has the particular formula hereinafter:

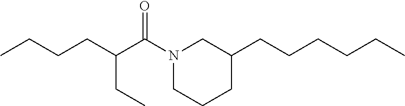 [SMILES: CCCCCCC1CCCN(C(=O)C(CC)CCCC)C1]

is synthesised using procedure B wherein 2-ethylhexanoyl chloride (890 μL, 5.10 mmol, 1.50 eq.) as the acid chloride, 3-hexylpiperidine hydrochloride as the cyclic amine salt (700 mg, 3.40 mmol, 1.00 eq.), triethylamine (1.84 mL, 13.6 mmol, 4.00 eq.) and DCM (12 mL) are used.

This yielded 990 mg (3.35 mmol) of EHPip3 as a yellow oil. Yield: 99%.

¹H NMR (CDCl₃, 400 MHz): δ (ppm): 4.52 (t, J=11.8 Hz, 1H, 100%), 3.89 (d, J=13.2 Hz, 1H, 100%), 2.99 (t, J=12.3 Hz, 1H, 50%), 2.69-2.51 (m, 2H, 100%), 2.32 (q, J=10.7, 12.3, 1H, 50%), 1.86 (d, J=12.3 Hz, 1H, 100%), 1.74-1.57 (m, 3H, 100%), 1.49-1.35 (m, 4H, 100%), 1.33-1.05 (m, 15H, 100%), 0.86 (m, 18H, 100%)

¹³C NMR (CDCl₃, 101 MHz): δ (ppm): 174.4 (C═O), 52.10, 52.06, 48.2, 46.7, 42.97, 42.94, 42.65, 42.63, 42.55, 42.52, 37.4, 36.35, 36.31, 34.05, 33.91, 32.81, 32.80, 32.6, 32.5, 31.9, 31.70, 31.68, 31.4, 30.2, 30.1, 30.02, 29.95, 29.7, 29.6, 26.9, 26.8, 26.4, 26.23, 26.19, 26.14, 26.11, 25.4, 23.04, 22.8, 22.7, 14.21, 14.16, 12.44 (50%), 12.42 (50%), 12.3 (50%), 12.2 (50%)

IR (cm⁻¹): 2957, 2928, 2872, 2855, 1639, 1460, 1439

HRMS (CI+): Theoretical exact mass for C₁₉H₃₈NO [M+H]⁺: 296.2953; experimental: 296.2973

### Synthesis of Monoamide PPPip3

The monoamide PPPip3, which has the particular formula hereinafter:

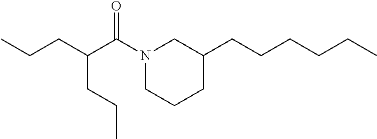 [SMILES: CCCCCCC1CCCN(C(=O)C(CCC)CCC)C1]

is synthesised using procedure A wherein 2-propylpentanoic acid is used as the carboxylic acid (2.93 mL, 18.4 mmol, 1.00 eq.), 3-hexylpiperidine hydrochloride as cyclic amine salt (3.00 g, 18.4 mmol, 1.00 eq.), HOBt (2.99 g, 22.1 mmol, 1.20 eq.), DCC (4.56, 22.1, 1.20 eq.) and 2-methyltetrahydrofuran (184 mL). N,N-diisopropylethylamine (or DIPEA—6.6 mL, 36.8 mmol, 2.00 eq.) is added initially to deprotonate 3-hexylpiperidine hydrochloride.

This yielded 4.03 g (13.63 mmol) of PPPip3 as a clear oil. Yield: 74%.

²H NMR (CDCl₃, 400 MHz): δ (ppm): 4.54 (t, J=12.1 Hz, 1H), 3.91 (d, J=13.3 Hz, 1H), 2.99 (td, J=12.7, 2.6 Hz, 1H, 50%), 2.74-2.51 (m, 2H), 2, 36-2.26 (m, 1H, 50%), 1.91-1.83 (m, 1H), 1.73-1.58 (m, 3H), 1.45-1.17 (m, 19H), 0.95-0.83 (m, 9H, —CH₃)

¹³C NMR (CDCl₃, 101 MHz): δ (ppm): 174.6 (C═O), 52.1, 48.2, 46.7, 43.0, 40.6, 40.5, 37.4, 36.3, 35.7, 35.6, 35.5, 34.1, 34.0, 31.9, 31.7, 31.4, 29.7, 29.6, 26.9, 26.8, 26.4, 25.4, 22.79, 22.76, 21.2, 21.1, 21.0, 20.9, 14.42, 14.40, 14.38, 14.2

IR (cm⁻¹): 2957, 2922, 2855, 1636, 1441

HRMS (CI+): Theoretical exact mass for C₁₉H₃₈NO [M+H]⁺: 296.2953, experimental: 296.2895

### Synthesis of Monoamide EHPip4

The monoamide EHPip4, which has the particular formula hereinafter:

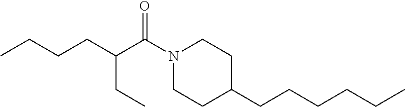 [SMILES: CCCCCCC1CCN(C(=O)C(CC)CCCC)CC1]

is synthesised using procedure B wherein 2-ethylhexanoyl chloride as the acid chloride (2.48 mL, 14.3 mmol, 1.05 eq.), 4-hexylpiperidine hydrochloride as the cyclic amine salt (2.80 g, 13.6 mmol, 1.00 eq.), triethylamine (4.60 mL, 34.0 mmol, 2.50 eq.) and DCM (136 mL) are used.

This yielded 3.51 g (12.5 mmol) of EHPip4 as a yellow oil. Yield: 92%.

¹H NMR (CDCl₃, 400 MHz): δ (ppm): 4.75-4.69 (m, 1H, 50%), 4.69-4.64 (m, 1H, 50%), 4.07-4.01 (m, 1H, 50%), 4.01-3.95 (m, 1H, 50%), 2.98 (t, J=12.4 Hz, 1H, 50%), 2.98 (t, J=12.4 Hz, 1H, 50%), 2.64-2.48 (m, 2H), 1.78-1.59 (m, 4H), 1.53-1.37 (m, 3H), 1.34-1.16 (m, 14H), 1.11-0.98 (m, 2H), 0.93-0.82 (m, 9H)

¹³C NMR (CDCl₃, 101 MHz): δ (ppm): 173.5 (C═O), 45.2, 41.6, 41.54, 41.45, 41.4, 35.6, 35.4, 32.5, 31.8, 31.6, 31.5, 31.0, 29.1, 29.0, 28.6, 25.7, 25.2, 25.1, 22.1, 22.0, 21.8, 13.24, 13.18, 11.4, 11.2

IR (cm⁻¹): 2953, 2926, 2868, 2856, 1628, 1429, 1246

HRMS (CI+): Theoretical exact mass for C₁₉H₃₈NO [M+H]⁺: 296.2953, experimental: 296.2963

### Synthesis of Monoamide EHPyr2

The monoamide EHPyr2, which has the particular formula hereinafter:

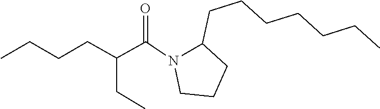 [SMILES: CCCCCCCC1CCCN1C(=O)C(CC)CCCC]

is synthesised using procedure B wherein 2-ethylhexanoyl chloride as the acid chloride (2.53 mL, 14.6 mmol, 1.20 eq.), 2-heptylpyrrolidine as the cyclic amine (2.17 g, 12.2 mmol, 1.00 eq.), triethylamine (2.46 mL, 18.3 mmol, 1.50 eq.) and DCM (165 mL) are used.

This yielded 4.42 g (15.7 mmol) of EHPyr2 as a yellow oil. Yield: 82%.

¹H NMR (CDCl₃, 400 MHz): δ (ppm): 4.17-4.08 (m, 1H, 67%), 3.89-3.80 (m, 1H, 33%), 3.61-3.39 (m, 2H, 100%), 2.42-2.31 (m, 1H, 100%), 2.03-1.78 (m, 4H), 1.72-1.39 (s, 5H), 1.35-1.16 (m, 15H), 0.92-0.83 (m, 9H)

¹³C NMR (CDCl₃, 101 MHz): δ (ppm): 175.14 (C═O, 16%), 175.06 (C═O, 16%), 174.73 (C═O, 33%), 174.71 (C═O, 33%), 57.65 (16%), 57.63 (16%), 57.3 (33%), 57.2 (33%), 47.0 (100%), 45.73, 45.71, 45.35, 45.32, 45.25, 35.65 (16%), 35.62 (16%), 33.48, 33.47, 33.4, 33.0, 32.6, 32.5, 32.0 (67%), 31.93 (16%), 31.91 (16%), 30.5, 30.23, 30.22, 30.19, 29.95, 29.92, 29.74, 29.72, 29.64, 29.56, 29.5, 29.4, 29.3, 29.2, 26.7, 26.7, 26.6, 26.5, 26.0, 25.9, 24.2, 23.2, 23.1, 23.0, 22.8, 22.2, 14.24, 14.21, 14.18, 12.70, 12.41, 12.18, 12.15

IR (cm⁻¹): 2957, 2924, 2864, 2855, 2636, 1419

HRMS (CI+): Theoretical exact mass for C₁₉H₃₈NO [M+H]⁺: 296.2953, experimental: 296.2962

### II—Extractive Properties of Monoamides of Formula (I)

Extraction tests are carried out using:
as organic phases: solutions comprising 1.2 mol/L of one of the following monoamides: EHPip2, EHPip3, PPPip3, EHPip4 and EHPyr2 in TPH; and
as aqueous phases: aqueous solutions comprising ≈10 g/L of uranium(VI), ≈200 kBq/mL of ²³⁹⁻²⁴⁰plutonium(IV) and nitric acid at a concentration of either 4 mol/L (to simulate the acidity likely to be exhibited by an aqueous solution resulting from the dissolution of a spent nuclear fuel in nitric acid) or 0.5 mol/L (to simulate the acidity that would be exhibited by an aqueous solution likely to be used to strip plutonium).

Each of these tests is carried out by bringing one of the organic phases into contact with one of the aqueous phases, in a tube and under stirring, for 30 min at 25° C. The O/A volume ratio used is 1. These phases are then separated from each other after centrifugation.

The uranium(VI) concentrations and the plutonium(IV) activities are measured in the organic and aqueous phases thus separated, respectively by plasma inductively coupled-atomic emission spectrometry (or ICP-AES) and by a spectrometry.

The distribution coefficients of uranium(VI) and plutonium(IV) as well as the U/Pu separation factors were determined in accordance with conventions in the field of liquid-liquid extractions, namely that:
the distribution coefficient of a metal element M, noted D_{M}, between two, respectively organic and aqueous, phases is equal to:

$${D}_{M} = \frac{{[ M ]}_{org .}}{{[ M ]}_{aq .}}$$

where:
[M]_{org.}=concentration of the metal element in the organic phase at extraction equilibrium (in mg/L); and
[M]_{aq.}=concentration of the metal element in the aqueous phase at extraction equilibrium (in mg/L);
   the separation factor between two metal elements M1 and M2, noted FS_{M1/M2}, is equal to:

$${FS}_{M 1 / M 2} = \frac{{D}_{M 1}}{{D}_{M 2}}$$

where:
D_{M1}=distribution coefficient of the metal element M1; and
D_{M2}=distribution coefficient of the metal element M2.

Table I hereinafter shows, for each monoamide tested, the distribution coefficients of uranium(VI), noted D_{U}, and of plutonium(IV), noted D_{Pu}, as obtained for the aqueous phases at 4 mol/L of HNO₃ and at 0.5 mol/L of HNO₃, as well as the separation factors U/Pu, noted FS_{U/Pu}, as obtained for the aqueous phases at 0.5 mol/L of HNO₃.

TABLE I

| | [C] | [HNO₃] | | | |
|---|---|---|---|---|---|
| Extractants tested | (mol/L) | (mol/L) | D_{U} | D_{Pu} | FS_{U/Pu} |
| 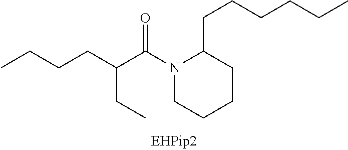 [SMILES: CCCCCCC1CCCCN1C(=O)C(CC)CCCC] | 1.2 | 4 0.5 | 9.7 0.31 | 1.8 0.014 | 22 |
| 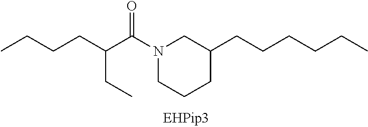 [SMILES: CCCCCCC1CCCN(C(=O)C(CC)CCCC)C1] | 1.2 | 4 0.5 | 15.1 0.42 | 5.1 0.027 | 16 |
| 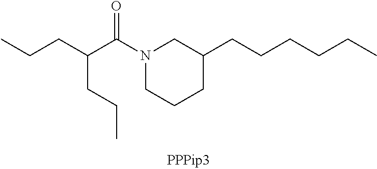 [SMILES: CCCCCCC1CCCN(C(=O)C(CCC)CCC)C1] | 1.2 | 4 0.5 | 14.8 0.54 | 5.3 0.029 | 19 |
| 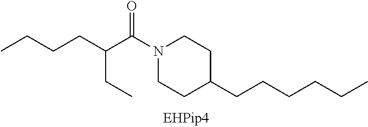 [SMILES: CCCCCCC1CCN(C(=O)C(CC)CCCC)CC1] | 1.2 | 4 0.5 | 8.4 0.29 | 3.2 0.022 | 13 |
| 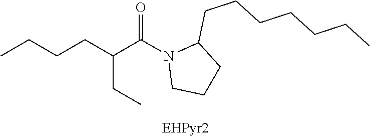 [SMILES: CCCCCCCC1CCCN1C(=O)C(CC)CCCC] | 1.2 | 4 0.5 | 16.8 1.07 | 14.1 0.032 | 33 |
| 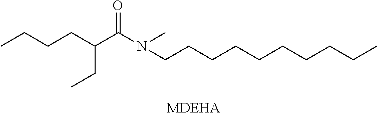 [SMILES: CCCCCCCCCCN(C)C(=O)C(CC)CCCC] | 1.2 | 4 0.5 | 9.1 0.32 | 4.3 0.018 | 18 |
| 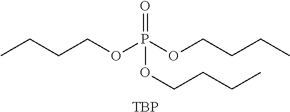 | 1.1 | 4 0.5 | 23.5 2.6 | 20.3 0.85 | 3.1 |

By way of comparison, also reported in this table are the results of tests carried out under the same operating conditions but using as the organic phases:
on the one hand, a solution comprising 1.2 mol/L of MDEHA (N-decyl-N-methyl-2-ethylhexanamide) provided in reference [2], in TPH, MDEHA being a dissymmetric monoamide free of a cyclic amine which has the same molecular formula (C₁₉H₃₈NO) as the monoamides tested and which comprises, like the monoamides EHPip2, EHPip3, EHPip4 and EHPyr2, a carbonyl group carrying a 1-ethylpentyl group; and
on the other hand, a solution comprising 1.1 mol/L of TBP in TPH since TBP is the solvent for the PUREX method.

Table I shows that all the monoamides tested co-extract uranium(VI) and plutonium(IV) at high acidity (D>1 for [HNO₃]=4 mol/L) and selectively strip plutonium (IV) at moderate acidity (D_{Pu}<0.05 for [HNO₃]=0.5 mol/L).

The piperidine ring monoamides EHPip3 and PPPip3 lead, at high acidity ([HNO₃]=4 mol/L), to distribution coefficients for uranium(VI) and plutonium(IV) that are higher than those obtained, at the same acidity and identical concentration in the organic phase, for MDEHA, while retaining good U/Pu selectivity at moderate acidity (FS_{U/Pu}>10 for [HNO₃]=0.5 mol/L).

However, the most interesting monoamide is the pyrrolidine ring monoamide EHPyr2. Indeed, this monoamide strongly extracts uranium(VI) and plutonium(IV) at high acidity ([HNO₃]=4 mol/L) while achieving a high U/Pu separation factor at moderate acidity (FS_{U/Pu}=33 for [HNO₃]=0.5 mol/L).

In addition, unlike monoamides such as MDEHA, which extract uranium(VI) more strongly than plutonium(IV), the monoamide EHPyr2 leads, at high acidity ([HNO₃]=4 mol/L), to similar distribution coefficients for uranium(VI) and plutonium(IV). These distribution coefficients are higher than those obtained, at the same acidity and identical concentration in the organic phase, for MDEHA. The U/Pu separation factor obtained, at moderate acidity ([HNO₃]=0.5 mol/L), for monoamide EHPyr2 is also higher than that obtained, at the same acidity, for MDEHA.

It should be noted that the uranium(VI) and plutonium(IV) distribution coefficients obtained for monoamide EHPyr2 at high acidity ([HNO₃]=4 mol/L are lower than those obtained at the same acidity for TBP. On the other hand, at moderate acidity ([HNO₃]=0.5 mol/L, the monoamide EHPyr2 has a U/Pu selectivity that is much higher than that of TBP, since it leads to a FS_{U/Pu} separation factor that is 10 times higher than that obtained for TBP at the same acidity.

### III—Uranium(VI) Loading Capacity of Monoamides of Formula (I):

Uranium (VI) loading capacity tests are carried out by subjecting an organic phase comprising 1.2 mol/L of monoamide EHPip3 in TPH to 5 successive contacts with an aqueous solution comprising 135 g/L uranium(VI) and 4 mol/L nitric acid.

Each contact is made in a tube under stirring, for 30 min at 25° C. and with an O/A volume ratio of 2, and is followed by centrifugation, separation of the aqueous and organic phases that have been brought into contact and measurement of the uranium(VI) concentration in the organic phase.

Table II hereinafter sets forth the uranium(VI) concentrations, noted [U]_{org} and expressed in g/L, as obtained in the organic phases after each of the 5 contacts.

TABLE II

| Contacts | [U]_{org} (g/L) |
|---|---|
| 1^{st} | 68 |
| 2^{nd} | 107 |
| 3^{rd} | 126 |
| 4^{th} | 141 |
| 5^{th} | 142 |

Further to the fact that no demixing (i.e. formation of a third phase) was noticed during the 5 contacts, Table II shows that it is possible to load an organic phase comprising a monoamide with 142 g/L of uranium(VI), which represents a high loading capacity, compatible with the development of a method for treating spent nuclear fuels.

### IV—Schematic Diagram of One Implementation of the Treatment Method of the Invention:

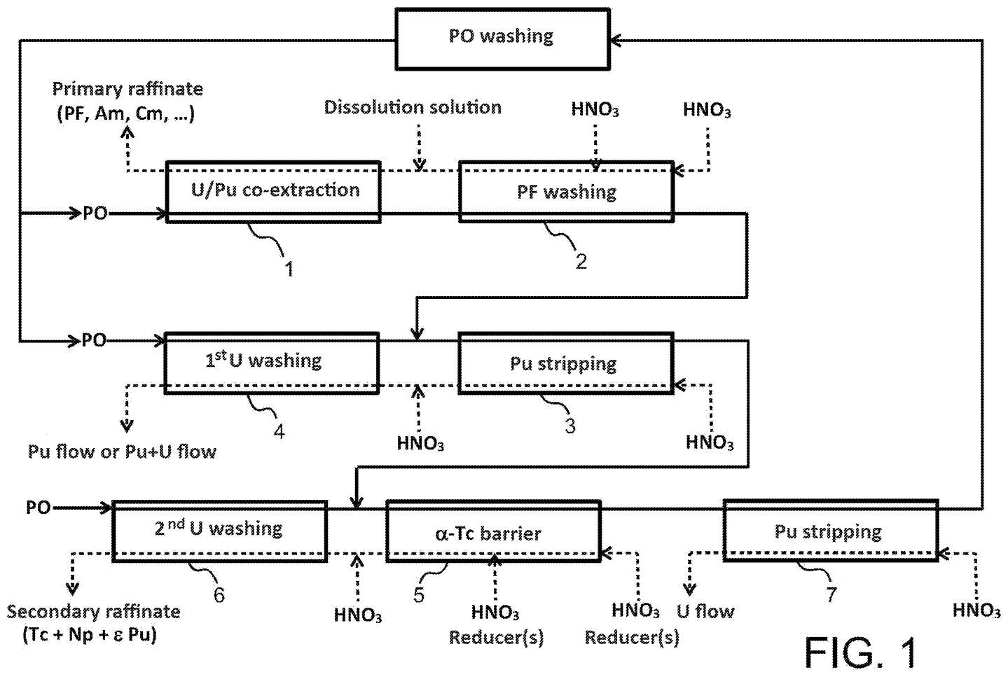

Reference is made to FIG. 1, which represents a schematic diagram of one implementation of a method for treating an aqueous solution resulting from the dissolution of a spent nuclear fuel in nitric acid in accordance with the invention.

As shown in the FIGURE, the method comprises 8 steps.

The first of these steps, noted “U/Pu co-extraction” in FIG. 1, is aimed at jointly extracting uranium and plutonium, the former in the +VI oxidation state and the latter in the +IV oxidation state, from the aqueous nitric solution resulting from the dissolution of a spent nuclear fuel.

Such a solution typically comprises from 3 to 6 mol/L of HNO₃, uranium(VI), plutonium(IV), minor actinides (americium, curium and neptunium), fission products (La, Ce, Pr, Nd, Sm, Eu, Gd, Mo, Ru, Tc, Rh, Ru, Pd, Y, Cs, Ba, Zr, Nb, . . . ) as well as a number of activation products such as chromium, manganese, iron, cobalt and nickel.

The “U/Pu co-extraction” step is carried out by circulating, in the extractor 1, the dissolution solution countercurrently to an organic phase (noted “PO” in FIG. 1) which comprises from 1 mol/L to 2 mol/L and, better still, from 1.1 mol/L to 1.3 mol/L, for example 1.2 mol/L, of a monoamide or a mixture of monoamides of formula (I), in solution in an organic diluent.

The organic diluent can especially be a straight or branched chain hydrocarbon such as n-dodecane, TPH or an isoparaffin such as that marketed under the trade name Isane™ IP 185T, preference being given to TPH.

The second step of the method, called “PF washing” in FIG. 1, aims at stripping, from the organic phase issued from the “U/Pu co-extraction”, the fraction of fission and activation products that was extracted together with the uranium(VI) and plutonium(IV) from the dissolution solution.

To do this, the “PF washing” step comprises one or more operations of washing the organic phase issued from the “U/Pu Co-extraction”, each washing operation being carried out by circulating this organic phase in the extractor 2, countercurrently to an aqueous nitric solution whose concentration can range from 1 mol/L to 6 mol/L of HNO₃ but is preferably from 4 mol/L to 6 mol/L of HNO₃ and, even better, from 4 mol/L to 5 mol/L of HNO₃ so as to facilitate the stripping of ruthenium and technetium.

If the “PF washing” step is carried out with one or more highly acidic aqueous solutions, i.e. typically equal to or greater than 3 mol/L of HNO₃, then this step also comprises deacidifying the organic phase, which is carried out by circulating this organic phase countercurrently to a weakly acidic nitric aqueous solution, i.e. comprising from 0.1 mol/L to 1 mol/L of HNO₃, such as an aqueous solution comprising 0.5 mol/L of HNO₃, in order to prevent too large an amount of acid from being carried to the extractor assigned to the third step, noted “Pu stripping” in FIG. 1, and impairing performance of this third step.

The “Pu stripping” step, which represents the first step of U/Pu partitioning, aims at stripping plutonium in the +IV oxidation state, and therefore without reduction of this plutonium, from the organic phase issued from the “PF washing”.

It is carried out by circulating this organic phase in the extractor 3 countercurrently to an aqueous solution comprising from 0.1 mol/L to 0.5 mol/L of HNO₃ and preferably using an O/A flow rate ratio greater than 1, preferably equal to or greater than 3 and, better still, equal to or greater than 5 so that the plutonium(IV) is stripped in a concentrating manner.

The stripping of plutonium(IV), which is carried out in the “Pu stripping” step, is accompanied with a stripping of a uranium(VI) fraction which is also present in the organic phase issued from the “PF washing”.

Thus, the fourth step of the method, noted “1st U washing” in FIG. 1 and representing the second step of the U/Pu partitioning, aims at extracting from the aqueous phase issued from “Pu Stripping”:
either all the uranium(VI) present in this aqueous phase, if the U/Pu partitioning is desired to lead to an aqueous solution comprising plutonium(IV) without uranium(VI), and to an organic solution comprising uranium(VI) without plutonium(IV);
or the amount of uranium(VI) making it possible to obtain, at the end of the “1st U washing”, an aqueous solution comprising uranium(VI) and plutonium(IV) in a pre-selected ratio, if it is desired that the U/Pu partitioning lead to an aqueous solution comprising a mixture of plutonium(IV) and uranium(VI) in this ratio and an organic solution comprising uranium(VI) without plutonium (IV).

In both cases, the “1st U washing” is carried out by circulating, in extractor 4, the aqueous phase issued from the “Pu stripping” countercurrently to an organic phase (especially noted as “PO” in FIG. 1) of identical composition to that of the organic phase used for the “U/Pu co-extraction”. The amount of uranium(VI) extracted is adjusted by varying, on the one hand, the O/A flow rate ratio and, on the other hand, the acidity of the aqueous phase, as the higher the organic phase/aqueous phase flow rate ratio and the higher the acidity of the aqueous phase, the better the extraction of uranium(VI). An addition of more or less concentrated HNO₃ to the aqueous phase circulating in the extractor 4 can therefore be provided depending on the acidity desired to be provided to this aqueous phase.

The fifth step, called “α-Tc barrier” in FIG. 1, aims at stripping, from the organic phase issued from the “Stripping Pu”, the technetium fraction which was extracted during the “Co-extraction U/Pu” but which was not stripped during the “PF washing”, in order to enhance the decontamination of this organic phase with respect to technetium.

It also enables the fraction of neptunium that was extracted during the “U/Pu co-extraction” and that followed the technetium to the “α-Tc barrier”, as well as any traces of plutonium(IV) that this organic phase is likely to still contain, to be stripped from the organic phase issued from the “Pu stripping”.

It is carried out by circulating, in extractor 5, the organic phase issued from the “Pu stripping” countercurrently to an aqueous nitric solution comprising from 0.1 mol/L to 3 mol/L of HNO₃ and, even better, 1 mol/L of HNO₃, as well as one or more reducing agents for reducing technetium—which is present in the organic phase in the +VII oxidation state—to technetium(IV), neptunium(VI) to neptunium(IV) or neptunium(V), and plutonium(IV) to plutonium(III) respectively, without reducing uranium(VI). For this type of acidity, technetium(IV) and neptunium(IV) are not extractable by monoamides of formula (I), while plutonium(III) is less extractable than plutonium(IV).

As reducing agents, uranous nitrate (or U(IV)), hydrazinium nitrate (or NH), hydroxylammonium nitrate (or NHA) and acetaldoxime, a hydroxyiminoalkanoic acid (6-hydroxyiminohexanoic acid for example) or a mixture thereof such as a U(IV)/NH, U(IV)/NHA or U(IV)/acetaldoxime mixture can thus be used, preference being given to a U(IV)/NH or U(VI)/NHA mixture. If necessary, gluconic acid can be added to the aqueous solution to decrease technetium(IV) reoxidation phenomena in the aqueous phase and thus limit consumption of reducing agent(s).

This step can be carried out at room temperature (i.e. at 20-25° C.) but is preferably carried out at a temperature of between 30° C. and 40° C. and, better still, 32° C. so as to promote the stripping kinetics of technetium(IV) while limiting technetium(IV) reoxidation phenomena in the aqueous phase and, therefore, the risk of having technetium, once stripped, re-extracted in the organic phase.

The sixth step, noted “2nd U washing” in FIG. 1, aims at extracting, from the aqueous phase issued from the “α-Tc Barrier”, the uranium(VI) which was stripped, together with technetium, in the previous step in order to prevent the “α-Tc Barrier” step from resulting in too great a loss of uranium(VI) in the aqueous phase.

It is carried out by circulating, in extractor 6, the aqueous phase issued from the “α-Tc Barrier” countercurrently to an organic phase (also noted as “PO” in FIG. 1) with a composition identical to that of the organic phases used for the “U/Pu Co-extraction” and the “1st U washing”, after acidifying this aqueous phase by adding concentrated nitric acid, especially at 10 mol/L, to promote extraction of uranium(VI).

The seventh step, noted “U stripping” in FIG. 1, aims at stripping uranium(VI) from the organic phase issued from the “α-Tc Barrier”.

It is carried out by circulating, in extractor 7, the organic phase issued from the “α-Tc barrier” countercurrently to a nitric aqueous solution comprising at most 0.5 mol/L and, even better, at most 0.05 mol/L of HNO₃, such as, for example, an aqueous solution comprising 0.01 mol/L of HNO₃. This step can be carried out at room temperature (i.e. at 20-25° C.) but is preferably carried out while hot (i.e. typically at a temperature of 40-50° C.) and using an O/A flow rate ratio greater than 1 so that uranium(VI) is extracted in a concentrating manner.

At the end of these 7 steps, the following are obtained:
two raffinates, which correspond to the aqueous phases leaving extractors 1 and 6 respectively and which comprise, for the first one, fission and activation products as well as americium and curium (“Primary raffinate” in FIG. 1) and, for the second one, technetium, neptunium and, optionally, traces of plutonium (“Secondary raffinate” in FIG. 1);
the aqueous phase leaving the extractor 4, which comprises either decontaminated plutonium(IV) or a mixture of decontaminated plutonium(IV) and uranium(VI) and which is called “Pu flow” or “Pu+U flow” depending on the case;
the aqueous phase leaving extractor 7, which comprises decontaminated uranium(VI) and which is called “U flow”; and
the organic phase leaving extractor 7, which no longer comprises either plutonium(IV) or uranium(VI) but which may contain a number of impurities and degradation products (formed by hydrolysis and radiolysis) of the extractant, which would have accumulated during the previous steps.

Thus, the eighth step, noted “PO washing” in FIG. 1, aims at regenerating this organic phase by subjecting it to one or more washes with a basic aqueous solution, for example a first wash with a 0.3 mol/L aqueous sodium carbonate solution, followed by a second wash with a 0.1 mol/L sodium hydroxide, followed by one or more washes with a nitric aqueous solution for reacidification, for example an aqueous solution comprising 2 mol/L HNO₃, each wash being carried out by circulating said organic phase in an extractor countercurrently to the aqueous washing solution.

As is visible in FIG. 1, the organic phase thus regenerated can then be returned to extractors 1 and 4 for reintroduction into the treatment cycle.

### REFERENCES CITED

[1] WO-A-2017/017207
[2] WO-A-2017/017193
[3] WO-A-2018/138441
[4] WO-A-2019/002788
[5] T. H. Siddall, Journal of Physical Chemistry 1960, 64, 12, 1863-1866
[6] H. Jing-Tian et al., Journal of Radioanalytical and Nuclear Chemistry 1999, 241, 215-217

What is claimed is:

1. A method for extracting uranium(VI) and/or plutonium(IV) from an acidic aqueous solution with a PH of less than 0, comprising contacting the acidic aqueous solution with an organic solution comprising a monoamide or a mixture of monoamides in an organic diluent, and then separating the aqueous and organic solutions from each other, wherein the monoamide(s) is (are) of formula (I): 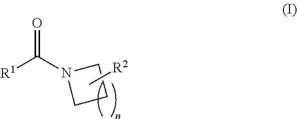
wherein:
n=1, 2 or 3;
R¹ is a straight or branched chain alkyl group comprising from 4 to 12 carbon atoms; and
R² is a straight or branched chain alkyl or alkoxy group, the chain of which is optionally interrupted one or more times by an oxygen atom and the total number of carbon atoms and, if applicable, of oxygen atoms is between 3 and 10.

2. (canceled)

3. The method of claim 1, wherein the organic solution comprises from 1 mol/L to 2 mol/L of the monoamide or of the mixture of monoamides.

4. The method of claim 1, wherein the acidic aqueous solution with a pH of less than 0 is an aqueous solution resulting from the dissolution of a spent nuclear fuel in nitric acid.

5. A method for totally or partially separating uranium(VI) from plutonium(IV) from an acidic aqueous solution with a pH of less than 0, comprising:
a) a co-extraction of uranium(VI) and plutonium(IV) from the acidic aqueous solution with a pH of less than 0, the co-extraction comprising at least one contacting of the aqueous solution with an organic solution S1 comprising a monoamide or a mixture of monoamides in an organic diluent, followed by a separation of the aqueous and organic solutions from each other, the monoamide(s) being of formula (I): 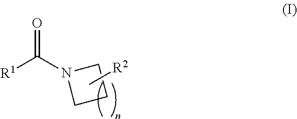
wherein:
n=1, 2 or 3;
R¹ is a straight or branched chain alkyl group comprising from 4 to 12 carbon atoms; and
R² is a straight or branched chain alkyl or alkoxy group, the chain of which is optionally interrupted one or more times by an oxygen atom and the total number of carbon and, if applicable, oxygen atoms is between 3 and 10;
b) a stripping of plutonium, in the +IV oxidation state, and of a fraction of the uranium(VI) from the organic solution issued from a), the stripping comprising at least one contacting of the organic solution with an acidic aqueous solution with a pH greater than 0, followed by a separation of the organic and aqueous solutions from each other; and
c) a re-extraction of all or part of the uranium(VI) fraction present in the aqueous solution issued from b), the re-extraction comprising at least one contacting of the aqueous solution with an organic solution S2 comprising the monoamide or the mixture of monoamides in the organic diluent, followed by a separation of the aqueous and organic solutions from each other;
whereby an aqueous solution comprising either plutonium(IV) without uranium(VI), or a mixture of plutonium(IV) and uranium(VI), and an organic solution comprising uranium(VI) without plutonium(IV) are obtained.

6. The method of claim 5, wherein the organic solutions S1 and S2 comprise from 1 mol/L to 2 mol/L of the monoamide or of the mixture of monoamides.

7. The method of claim 5, wherein the acidic aqueous solution with a pH less than 0 is an aqueous solution resulting from the dissolution of a spent nuclear fuel in nitric acid, while the acidic aqueous solution with a pH greater than 0 is a solution comprising from 0.1 mol/L to 0.5 mol/L of nitric acid.

8. The method of claim 5, wherein n=2 or 3.

9. The method of claim 5, wherein:
R¹ is a straight or branched chain alkyl group comprising from 6 to 10 carbon atoms; and/or
R² is a straight or branched chain alkyl group comprising from 4 to 10 carbon atoms.

10. The method of claim 9, wherein the monoamide is:
N-(2-heptylpyrrolidinyl)-(2-ethyl)hexanamide,
N-(2-hexylpiperidinyl)-(2-ethyl)hexanamide,
N-(3-hexylpiperidinyl)-(2-ethyl)hexanamide,
N-(4-hexylpiperidinyl)-(2-ethyl)hexanamide, or
N-(3-hexylpiperidinyl)-(2-propyl)pentanamide.

11. A method for treating in one cycle an aqueous solution resulting from a dissolution of a spent nuclear fuel in nitric acid, the aqueous solution comprising uranium(VI), plutonium(IV), americium(III), curium(III) and fission and activation products including ruthenium and technetium, the cycle comprising:
a) a co-extraction of uranium(VI) and plutonium(IV) from the aqueous solution, the co-extraction comprising at least one contacting, in an extractor, of the aqueous solution with an organic solution S1 comprising a monoamide or a mixture of monoamides in an organic diluent, followed by a separation of the aqueous and organic solutions from each other, the monoamide(s) being of formula (I): 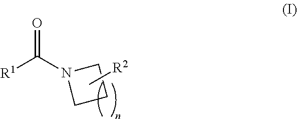
wherein:
n=1, 2 or 3;
R¹ is a straight or branched chain alkyl group comprising from 4 to 12 carbon atoms; and
R² is a straight or branched chain alkyl or alkoxy group, the chain of which is optionally interrupted one or more times by an oxygen atom and the total number of carbon atoms and, if applicable, of oxygen atoms is between 3 and 10;
b) a decontamination of the organic solution issued from a) with respect to americium(III), curium(III) and fission and activation products, the decontamination comprising at least one contacting, in an extractor, of the organic solution with an aqueous solution comprising from 0.5 mol/L to 6 mol/L of nitric acid, followed by a separation of the organic and aqueous solutions from each other;
c) a partitioning of the uranium(VI) and plutonium(IV) present in the organic solution issued from b) into an aqueous solution comprising either plutonium(IV) without uranium(VI), or a mixture of plutonium(IV) and uranium(VI), and an organic solution comprising uranium(VI) without plutonium(IV), the partitioning comprising:
c₁) a stripping of plutonium, in the +IV oxidation state, and of a fraction of the uranium(VI) from the organic solution issued from b), the stripping comprising at least one contacting, in an extractor, of the organic solution with an aqueous solution comprising from 0.1 mol/L to 0.5 mol/L of nitric acid, followed by a separation of the organic and aqueous solutions from each other;
c₂) a re-extraction of all or part of the uranium(VI) fraction present in the aqueous solution issued from c₁), the re-extraction comprising at least one contacting, in an extractor, of the aqueous solution with an organic solution S2 comprising the monoamide or the mixture of monoamides in the organic diluent, followed by a separation of the aqueous and organic solutions from each other;
d) a decontamination of the organic solution issued from c₁) with respect to technetium, the decontamination comprising:
d₁) a stripping of technetium, in the +IV oxidation state, from the organic solution issued from c₁), the stripping comprising at least one contacting, in an extractor, of the organic solution with an aqueous solution comprising from 0.1 mol/L to 3 mol/L of nitric acid and at least one reducing agent capable of reducing technetium from the +VII oxidation state to the +IV oxidation state, followed by a separation of the organic and aqueous solutions from each other;
d₂) a re-extraction of the uranium(VI) fraction present in the aqueous solution issued from d₁), the re-extraction comprising at least one contacting, in an extractor, of the aqueous solution with an organic solution S3 comprising the monoamide or the mixture of monoamides in the organic diluent, followed by a separation of the aqueous and organic solutions from each other;
e) a stripping of uranium(VI) from the organic solution issued from d₁), the stripping comprising at least one contacting, in an extractor, of the organic solution with an aqueous solution comprising at most 0.05 mol/L of nitric acid, followed by a separation of the organic and aqueous solutions from each other; and
f) optionally, a regeneration of the organic solution issued from e);
whereby a first and a second aqueous solution decontaminated with respect americium(III), curium(III) and fission and activation products are obtained, the first aqueous solution comprising either plutonium(IV) without uranium(VI), or a mixture of plutonium(IV) and uranium(VI), and the second aqueous solution comprising uranium(VI) without plutonium(IV).

12. The method of claim 11, wherein the organic solutions S1, S2 and S3 comprise from 1 mol/L to 2 mol/L of the monoamide or mixture of monoamides.

13. The method of claim 11, wherein the aqueous solution of b) comprises from 4 mol/L to 6 mol/L of nitric acid before being contacted with the organic solution.

14. The method of claim 11, wherein the contacting of the organic and aqueous solutions in the extractor of c₁) comprises circulating the organic and aqueous solutions with a ratio of a flow rate of the organic solution to a flow rate of the aqueous solution greater than 1.

15. The method of claim 11, wherein the reducing agent is uranous nitrate, hydrazinium nitrate, hydroxylammonium nitrate, acetaldoxime, hydroxyiminoalkanoic acid, or a mixture thereof.

16. The method of claim 11, wherein:
the extractor of d₁) is heated to a temperature of 30° C. to 40° C. and/or
the extractor of e) is heated to a temperature of 40° C. to 50° C.

17. The method of claim 11, wherein the contacting of the organic and aqueous solutions in the extractor of e) comprises circulating the organic and aqueous solutions with a ratio of the flow rate of the organic solution to the flow rate of the aqueous solution greater than 1.

18. The method of claim 11, wherein the organic solution issued from e) is regenerated and then divided into a first and a second fraction, the first fraction forming the organic solution S1 and the second fraction forming the organic solution S2.

19. The method of claim 11, wherein n=2 or 3.

20. The method of claim 11, wherein:
R₁ is a straight or branched chain alkyl group comprising from 6 to 10 carbon atoms; and/or
R₂ is a straight or branched chain alkyl group comprising from 4 to 10 carbon atoms.

21. The method of claim 11, wherein the monoamide is:
N-(2-heptylpyrrolidinyl)-(2-ethyl)hexanamide,
N-(2-hexylpiperidinyl)-(2-ethyl)hexanamide,
N-(3-hexylpiperidinyl)-(2-ethyl)hexanamide,
N-(4-hexylpiperidinyl)-(2-ethyl)hexanamide, or
N-(3-hexylpiperidinyl)-(2-propyl)pentanamide.

22. Monoamide, which is:
N-(2-heptylpyrrolidinyl)-(2-ethyl)hexanamide,
N-(2-hexylpiperidinyl)-(2-ethyl)hexanamide,
N-(3-hexylpiperidinyl)-(2-ethyl)hexanamide,
N-(4-hexylpiperidinyl)-(2-ethyl)hexanamide, or
N-(3-hexylpiperidinyl)-(2-propyl)pentanamide.
